(19) United States
(12) Patent Application Publication
(10) Pub. No.: US 2006/0084916 A1
(43) Pub. Date: Apr. 20, 2006

(54) Self-destruction syringe

(76) Inventor: Taming Lo, Taipei (TW)

Correspondence Address: PRO-TECHTOR INTERNATIONAL, 20775 Norada Court, Saratoga, CA, 95070-3018, US

(21) Appl. No.: 10/890,956

(22) Filed: Jul. 14, 2004

(51) Int. Cl. A61M 5/00

(52) U.S. Cl. 604/110

(57) ABSTRACT

The present invention relates to a self-destruction syringe having a metal element formed inside a Luer-Lock tip of a hollow barrel to stab a rubber bulb of a plunger after the injection, which disabling the plunger to draw medicament solution again, thereby reaching the self-destruction effect and also preventing the syringe from being re-used.

### BACKGROUND OF THE INVENTION

(a) Field of the Invention

The present invention relates to a syringe with a metal element formed in a Luer-Lock tip, which stabs in a rubber bulb of a plunger after the injection, causing a leakage of the plunger, thereby disabling the plunger to draw medicament and reaching a mandatory self-destruction function.

(b) Description of the Prior Art

A conventional self-destruction syringe includes a constraint device to restrain a plunger being pulled back to avoid the syringe being re-used. Due to high production cost, the conventional self-destruction syringe is yet to be widely adopted by developing and un-developed countries to replace a conventional syringe.

### SUMMARY OF THE INVENTION

The purpose of the present invention is to form a mandatory self-destruction mechanism in a syringe, which can no longer draw medicament after use, thereby preventing the syringe from being re-used. With the production cost almost the same as that of a conventional syringe, the present invention has the potential to replace the conventional syringe.

To enable a further understanding of the said objectives and the technological methods of the invention herein, the brief description of the drawings below is followed by the detailed description of the preferred embodiments.

### BRIEF DESCRIPTION OF THE DRAWING

FIG. 1 shows a prospective view of the present invention.

FIG. 2 shows an exploded elevational view of the present invention.

FIG. 3 shows a cross-sectional view of the present invention.

FIG. 4 shows a cross-sectional view of the present invention before use.

FIG. 5 shows a cross-sectional view of the present invention after use.

FIG. 6 shows a cross-sectional view of the present invention when a plunger being pulled back after the injection.

### DETAILED DESCRIPTION OF THE PREFERRED EMBODIMENTS

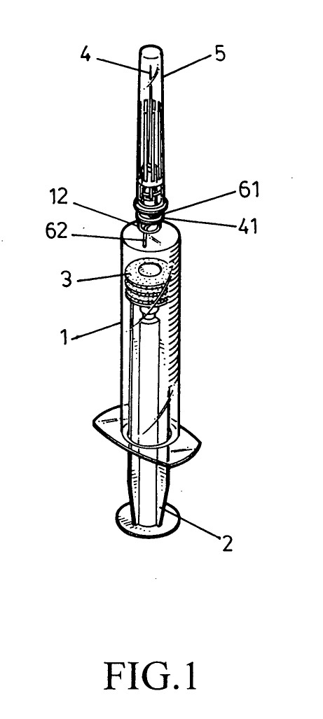

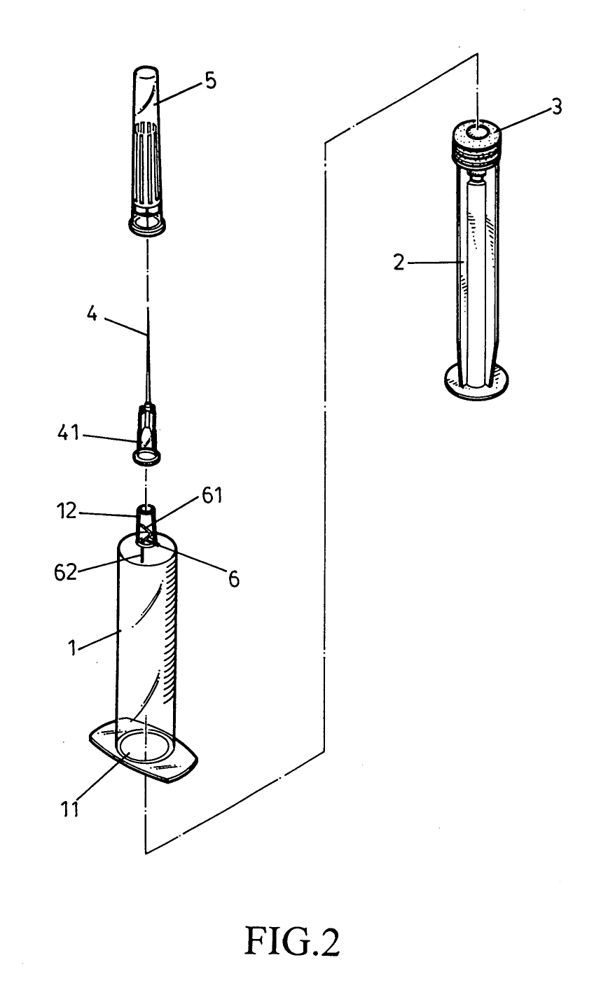

Referring to FIG. 1˜3. The present invention includes a hollow barrel 1, a plunger 2, a rubber bulb 3, a needle 4, and a tip protector 5, wherein the rubber bulb 3 formed in the front of the plunger 2 inserting into a hollow 11 of the hollow barrel 1, the plunger 2 can be moving forward or afterward inside the hollow barrel 1, a needle hub 41 formed at the bottom of the needle 4 capping on a Luer-Lock tip 12 of the hollow barrel 1, and the tip protector 5 can be capped on the needle hub 41, with characteristics as follow,

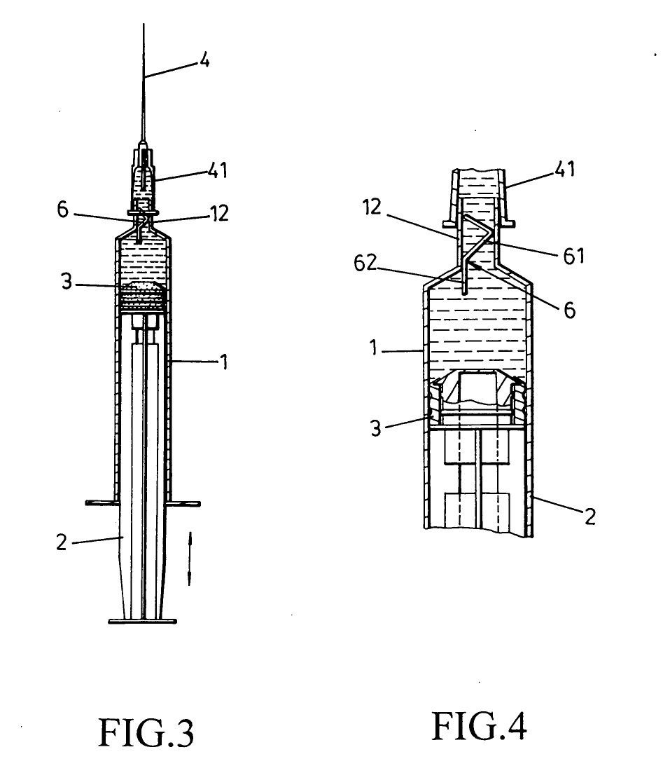

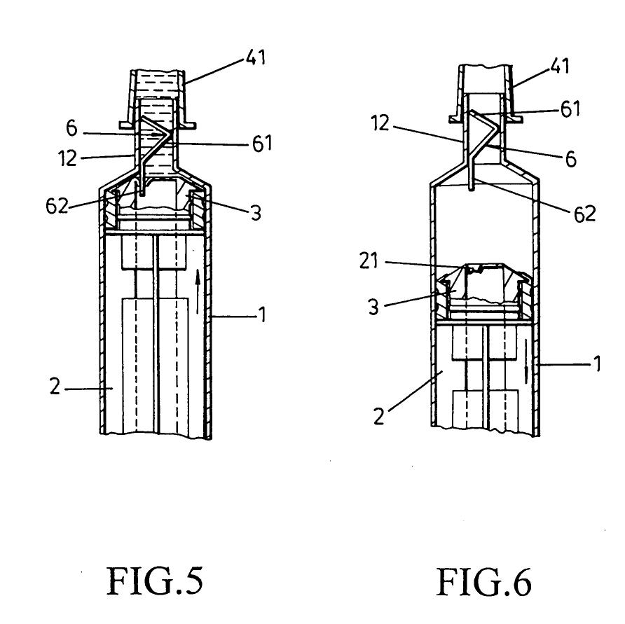

Referring to FIG. 3. Inside the Luer-Lock tip 12 of the hollow barrel 1, a metal element 6 formed, with one end 61, being in slanted L-shape, fixed at the Luer-Lock tip 12, and the other end 62, being a straight portion, protruding inside the hollow barrel 1. Referring to FIG. 4. The rubber bulb 3, being undamaged before using, contacts closely with the inner surface of the hollow barrel 1 such that a suction force formed to draw medicament solution, when pulling the plunger 2 backward. Referring to FIGS. 5 and 6. When performing the injection, the plunger 2 is to be pushed forward till the end, causing the straight end 62 of the metal element 6 stabbing into the rubber bulb 3 to form a hole 21, thereby disabling the plunger 2 to draw medicament solution again and reaching a self-destruction function.

In summary, the present invention is to add the metal element inside the Luer-Lock tip such that the rubber bulb of the plunger will be stabbed by the metal element, which disabling the syringe to be re-used, thereby reaching a self-destruction effect and also preventing the syringe from being re-used.

It is of course to be understood that the embodiment described herein is merely illustrative of the principles of the invention and that a wide variety of modifications thereto may be effected by persons skilled in the art without departing from the spirit and scope of the invention as set forth in the following claims.

What is claimed is:

1. A self-destruction syringe comprising
a hollow barrel, wherein a hollow formed at one end and a Luer-Lock tip formed at the other end;
a plunger, being formed inside the hollow barrel;
a rubber bulb, being formed in the front of the plunger;
a needle, being formed on a needle hub and capped by a tip protector;
a metal element, wherein one end, being in slanted L-shape, being fixed at the Luer-Lock tip and the other end, being a straight portion, protruding inside the hollow barrel.
